(19) United States
(12) Patent Application Publication
(10) Pub. No.: US 2006/0167096 A1
(43) Pub. Date: Jul. 27, 2006

(54) Livestock products with an increased ppar/rxr heterodimer activator level

(76) Inventor: Luc De Keyser, Denderleeuw (BE)

Correspondence Address: SUGHRUE MION, PLLC, 2100 PENNSYLVANIA AVENUE, N.W., SUITE 800, WASHINGTON, DC, 20037, US

(21) Appl. No.: 10/561,810

(22) PCT Filed: Jun. 23, 2004
(86) PCT No.: PCT/EP04/51205

§ 371 Date: Dec. 21, 2005

(30) Foreign Application Priority Data
Jun. 26, 2003 (WO) PCT/BE03/00112

(51) Int. Cl. A61K 31/20

(52) U.S. Cl. 514/558

(57) ABSTRACT

In the non-therapeutic method livestock animals, used in agri- or aquaculture for producing livestock products, are made to ingest at least one product comprising a PPAR/RXR heterodimer activator and/or a precursor thereof over such a period of time and in such an amount that the PPAR/RXR heterodimer activator is accumulated in the livestock animal. In this way, livestock products such as meat, milk and eggs having an increased PPAR/RXR heterodimer activator level can be obtained. The PPAR/RXR heterodimer activator is phytanic acid, a metabolite of phytanic acid, a derivative of phytanic acid or of said metabolite, or a combination thereof. In order to accumulate the PPAR/RXR heterodimer activator in the livestock animal, a predetermined minimum amount of said product, in particular of phytol, is given to the livestock animals over at least one period of at least three days.

The present invention relates to a non-therapeutic method for achieving an increased level of at least one PPAR/RXR heterodimer activator in a livestock product for human consumption, in particular in skeletal meat, milk and/or eggs, in which method livestock animals, used in agri- or aquaculture for producing the livestock product, are made to ingest at least one product comprising said PPAR/RXR heterodimer activator and/or a precursor thereof which is metabolised by the livestock animals into said PPAR/RXR heterodimer activator, over such a period of time and in such an amount that the PPAR/RXR heterodimer activator is accumulated in the livestock animal so that said increased PPAR/RXR heterodimer activator level is achieved in the livestock product.

An example of a PPAR/RXR heterodimer activator is conjugated linoleic acid (CLA). EP-A-1 106 077 discloses a method wherein a feed comprising extruded linseed is given to cows. This feed is intended to achieve milk having a particular content of saturated and unsaturated fatty acids and, in particular, an elevated CLA content. Other methods wherein the level of CLA in ruminant livestock products is enhanced through altering the dietary composition in the feeds such that more CLA is produced are disclosed in [Offer 1998] and in [Chilliard 2000]. CLA can also be supplemented directly to the feeds of other livestock such as pigs [Ostrowska 1999], poultry and fish in order to achieve enhanced levels of CLA in pork, chicken meat, eggs and fish meat.

In the following table, a number of references disclosing CLA levels obtained by supplementing the feed of livestock animals with CLA are given. It displays per reference, the product targeted, the maximum level of CLA in the diet by weight and the maximum level of CLA found in that product as a percent of total fatty acids.

| | | max in | |
|---|---|---|---|
| Reference | product | diet | max of TFA |
| Chamruspollert | egg yolk | 5% | 11.2% |
| 1999 | | | |
| Shafer 2001 | egg yolk | 2% | 7.95% |
| Raes 2002 | egg yolk | 3% | 5.3% |
| Szymczyk 2001 | Chicken meat | 1.5% | 10.27% |
| Choi 1999 | Carp | 1% | 13% |
| Twibell 2000 | striped bass | 0.6% | 8.1% |
| Twibell 2001 | yellow perch | 0.6% | 2.92% |
| Ramsey 2001 | lean pork | 1.4% | 3.2% |
| | | | (up to 55 kg) |
| Thiel-Cooper | lean pork | 1% | 0.7% |
| 2001 | | | (>100 kg) |
| Joo 2002 | lean pork | 5% | 1.6% |
| | | | (>100 kg) |

CLA is a fatty acid that has generated a lot of interest with respect to health since the discovery that grilled minced beef could inhibit carcinogenesis [Ha 1987]. During the last 15 years, numerous other physiological properties have been attributed to CLA beside it being anticarcinogenic [Belury 2002], including action as an antiadipogenic [Smedman 2001], antidiabetogenic [Houseknecht 1998, Ryder 2001] and antiatherosclerotic [Wilson 2000] agent. Furthermore CLA has effects on bone formation [Li 1999] and the immune system [Sugano 1998].

CLA stands for a group of positional and stereo-isomers of conjugated octadecadienoic acid, a fatty acid doubly unsaturated in positions separated by just one single bound and whereby one of the double bounds is in trans and the other in the cis steomeric configuration.

The natural source of CLA in foods is almost exclusively from ruminant livestock products like beef, lamb and dairy. The predominant isomer is c9t11-CLA. Several other isomers are also found such as t7,c9-CLA, c11t13-CLA, c8t10-CLA and t10c12-CLA [Fritsche 1999].

The synthetic production of CLA is usually based on an alkalinisation of a linoleic acid substrate. This process generates predominantly two isomers in roughly equal proportions: c9t11-CLA and t10c12-CLA [Reaney 1999]. The majority of the studies on CLA were performed with such a CLA isomer mixture.

In the general population, the intake of CLA has been estimated to vary widely between 15-659 mg/day [Park 1999]. As amounts as small as 0.5% of diet have been shown to alter expression of genes and impact conditions such as carcinogenesis, obesity, diabetes, and atherosclerosis in, mostly, animal studies, it is quite likely that these amounts taken over longer periods have similar benefits for the specific human subgroups.

The mechanisms underlying the beneficial effects of CLA are slowly but surely being elucidated. One complicating factor is that the different CLA isomers seem to have some common and some different courses of action [Pariza 2001].

One line of action is based on the mediation of the peroxisome proliferator-activated receptor (PPAR). These are orphan nuclear receptors that require a dimerisation with a retinoid-X receptor (RXR) that when activated, straddle the peroxisome proliferator response elements (PPRE's) on the DNA to trigger the transcription of a particular set of genes. PPARs come in three families alpha, beta (or delta) and gamma.

PPAR alpha is a PPAR family that is involved in the metabolism of fatty acids and lipoproteins. Synthetic activators of PPAR alpha include the lipid-lowering fibrates. These have been used for years in clinical medicine to treat dyslipidemias. In addition, PPAR alpha activation improves insulin sensitivity and decreases inflammation in the vascular walls and thrombi. Each of these is an important factor in the onset, progression and complications of atherosclerosis. Furthermore, PPAR alpha ligands have been shown to prevent the induction and halt the progression of certain cancers in cell line and animal models. It has been shown that CLA is an agonist of PPAR alpha [Moya-Camarena 1999].

PPAR gamma is another PPAR family that is involved with adipogenesis and lipid metabolism. Thiazolidinediones (TZD) are potent insulin sensitizers used to treat type II diabetes. They were found to be synthetic ligands of PPAR gamma. In addition, PPAR gamma stimulation inhibits the production of a number of cytokines that are involved in promoting inflammation. Furthermore, the activation of PPAR gamma has been shown to prevent the induction of a number of cancers by promoting cell differentiation and stimulating apoptosis. It has been shown conclusively that CLA is an agonist of PPAR gamma [Houseknecht 1998, Yu 2002].

A second mode of action is through the inhibition of particular enzymes that elongate [Chuang 2001] and desaturate [Park 2000] fatty acids. Although the impact of a mix of isomers of CLA, or of the individual isomers are not fully elucidated yet, it appears that CLA, through this mechanism, influences the level and character of cytokines derived from the LOX and COX fatty acid oxidation pathways [Urquhart 2002] and, consequently, impacts inflammation and blood clotting behavior.

Given the important potential health benefits of CLA, the required daily allowance has been calculated to be between 1.5 g and 3 g per day [Decker 1995]. As the present level of CLA in the diet is about three to ten times less than required, it became necessary to devise ways to supplement CLA in the human diet.

Although CLA is a compound with a unique position in the human food chain and with interesting properties and potential for health promotion, it presents a number of important hurdles for its generalized supplementation in the human diet:

CLA is represented by a variety of isomers exposing different and sometimes even opposite activities.

The mechanisms of action of CLA are varied and influencing several different pathways simultaneously making it hard to elucidate the relative importance of each.

As CLA joins the same pathways as linoleic acid and linoleic acid is a key-precursor for a couple of families of cytokines involved in the delicate balance in inflammation and clotting, the effect of CLA derived cytokines on this balance is worrisome.

CLA supplementation decreases to a certain degree the effect of endogenous desaturases [Lee 1998]. This causes a serious shift in the fatty acid profile of foods from animal origin towards more of the less desirable saturated fatty acids.

The large majority of studies have been using a mix of CLA isomers, complicating the interpretation of the mechanisms of action even more and casting serious doubt on any extrapolation.

CLA is an unsaturated fatty acid and thus prone to oxidation [Hamalainen 2002], for example during cooking. Although CLA is relatively stable during storage and processing, the toxicological profile of its degradation products in foods remains elusive. In vivo, CLA has been shown to be reactive enough to, at least, induce lipid peroxidation products that are markers of arteriosclerosis [Basu 2000, Riserus 2002].

The natural sources of CLA in the food chain are bacteria detoxifying a linoleic acid overload [Fukuda 2002]. The complete chemical synthesis of CLA is possible but not well established. The industrial production of CLA from plant based oils generates an unnatural mix of isomers [Saebo 2001]. Moreover, the isomer specific purification of CLA is far from trivial.

In addition, it has been shown that CLA is produced endogenously from the trans monoene vaccenic acid [Adlof 2000] [Loor 2002]. This puts into question the necessity to supplement foods with CLA, in particular with isomers that are not generated by the mammalian organism itself.

Furthermore, the association between CLA and a trans fatty acid like vaccenic acid complicates the interpretation of the conflict between the potential beneficial effects of CLA and the generally accepted noxious effects of trans fatty acids.

Lastly, upto now there is little data about the effect of CLA in acute toxic and long-term lower level overload conditions.

An object of the present invention is to provide a new method for producing livestock products for human consumption which enables to achieve livestock products which also have interesting properties and potential for health promotion due to the presence of an increased level of a PPAR/RXR heterodimer activator but wherein a PPAR/RXR heterodimer activator or a precursor thereof different from CLA is used so that at least a number of the drawbacks of CLA indicated hereabove are obviated.

To this end, the method according to the present invention is characterised in that said PPAR/RXR heterodimer activator is phytanic acid, a metabolite of phytanic acid, a derivative of phytanic acid or of said metabolite, or a combination thereof and, in order to accumulate the PPAR/RXR heterodimer activator in the livestock animal, a predetermined amount of said product is given to the livestock animals over at least one period of at least three days, during which the livestock animals ingest a total amount of F kg feed dry weight, which predetermined amount of said product contains at least 5×F meq, preferably at least 10×F meq, and more preferably at least 15×F meq of said PPAR/RXR heterodimer activator and/or precursor thereof.

Phytanic acid (PhA) is the common name for tetramethylhexadecanoic acid, a saturated fatty acid with four methyl branches. The PhA catabolism has been studied extensively for the last forty years, primarily, to explain the pathophysiology of Refsum's disease, a rare genetic disorder affecting the peroxisome metabolism [Verhoeven 2001]. In the late seventies, it was found that adhering to a low PhA diet could prevent the noxious accumulation of PhA and, soon, the PhA levels of foodstuffs were measured and specific dietary tables were established [Masters-Thomas 1980].

In the human diet, the most important sources of PhA are rumen products, such as from beef and dairy products, and fish products such as from herring, sardines and mackerel and the like. The PhA in these animals is the result of the uptake of phytol released during the breakdown of chlorophyll. Phytol is converted to PhA in the liver. PhA itself is broken down in pristanic acid (PrA) through an alpha-oxidation and subsequently in trimethyltetradecanoic acid (TMTD) through a beta-oxidation. Both these oxidations and the following two beta-oxidations occur in the peroxisome. The next ones occur in the mitochondrium.

In the rumen of ruminants, the chlorophyll contained in the forage grasses is broken down during the fermentation in the gut. The fish, on the other hand, obtain phytol by ingesting zooplankton that has been feeding on the phytoplankton. It is not generally known which microorganisms are responsible for hydrolyzing chlorophyll, neither in the rumen nor in the plankton.

After [Van den Branden 1986] noted that dietary phytol induced the proliferation of hepatic peroxisomes in adult mice, cell research showed that PhA is a ligand of RXR [Kitareewan 1996] and subsequently it was identified as a potent activator of PPAR alpha in physiologic concentrations [Ellinghaus 1999]. These characteristics point towards a number of promising human health claims such as against atherosclerosis [Pineda Torra 1999], non-insulin dependent diabetes [Lenhard 2001] and cancer [Roberts-Thomson 2000]. As CLA had also been found to be an agonist of PPAR alpha [Moya-Camarena 1999] and PPAR gamma [Houseknecht 1998], some potential health benefits of CLA were hypothesized to pertain also to PhA.

In 2001, McCarty hypothesised that supplementing the human diet with hydrolysed chlorophyll at a dosage of 0.5% of the diet weight in free phytol could be an effective prevention and treatment of non-insulin dependent diabetes [McCarty 2001]. He based his argument on the finding that cell research showed that some early phytol metabolites are a ligand of RXR [Kitareewan 1996] and that the PPARgamma/RXR heterodimer was suggested as a target for treating diabetes [Mukherjee 1997]. As CLA was found to be an agonist of PPAR gamma [Houseknecht 1998], some health benefit claims of CLA could possibly extend also to phytol and its metabolites.

Although the potential beneficial effects of PhA are known and although direct supplementation of the human diet with PhA or its precursor phytol has already been disclosed in [McCarty 2001], EP-A-1 177 789 and in WO-A-9709039, nobody has suggested up to the present invention any feeding strategy to enhance the level of phytol or its metabolites or derivatives thereof in food products of animal origin for human consumption.

Compared to the above described disadvantages of the prior art methods wherein the human food is supplemented with CLA, the method according to the invention offers however the following advantages as a result of the use of PhA (or metabolites or derivatives thereof) as PPAR/RXR heterodimer activator:

As PhA is completely saturated it does not present itself in different isomeric configurations, exposing possibly different activities like CLA isomers do.

Although it cannot be excluded that PhA has other more subtle mechanisms of action, its main effect is evidently through its agonistic effect on the PPAR/RXR system.

Although it cannot be excluded that PhA metabolises in other minor pathways, its main catabolic pathway has been completely elucidated in minute detail, together with a list of known genetic mutations that perturb this pathway.

Although only relatively few PhA supplementation studies have been performed, their interpretation is not complicated by a mixture of compounds with possible opposing activities like with CLA.

As PHA is fully saturated there is no inherent problem of oxidation. This means that the compound is not only stable during storage, processing and heating, but that also we do not expect in vivo reactions such as lipid peroxidation that cast doubt over CLA as a potential healthy supplement.

The natural source of PhA is the chlorophyll used in plants and algae. The complete synthetic chemical synthesis is well established [Eldjarn 1966] and is the preferred industrial method to produce precursors of vitamins such a vitamin K and vitamin E. The industrial production of PhA from plant-based material is also relatively trivial.

As there is no endogenous production of PhA from any lower level precursor in the animal kingdom, all PhA in the organism is of dietary origin. This eliminates the uncertainty about influences of other precursors like trans vaccenic acid does with in CLA studies.

As Refsum patients have been studied thoroughly, we have extensive information about the metabolic effects of long term toxic doses.

Direct supplementation of the human or animal diet with phytol or phytanic acid has already been disclosed in the prior art but only for therapeutic purposes. EP-A-1 177 789 discloses the therapeutic use of PhA or phytol for the treatment or prevention of diabetes whilst in WO-A-9709039, PhA is described to be a vitamin, more particularly vitamin F, which can be used for treating vitamin F deficiency. Vitamins are however used in very small, trace concentrations and are never meant to accumulate in tissues. Moreover, also in EP-A-1 177 789, the phytanic acid or phytol is administered in relatively small daily doses, more particularly in daily doses of between 0.1 and 50 mg/kg body weight, and usually of between 0.5 and 40 mg/kg body weight. Although EP-A-1 177 789 mentions the use of phytol or phytanic acid for preventing or treating diabetes in humans or animals, it does not teach any specific animals and a skilled person would not use it for livestock animals since these animals do not suffer from diabetes that warrants treatment. Moreover, EP-A-1 177 789 does not teach to supplement feed with phytol or phytanic acid to achieve an accumulation of phytanic acid in the livestock products, no tissue concentrations being indicated at all.

In other prior art documents, the accumulation of PhA in certain tissues has been mentioned.

Lough [Lough 1977] has noted the possible effect of natural feeds (containing chlorophyll) on the level of PhA in the liver, kidney, heart, brain, omental fat, plasma and milk in a dozen of cows and steers However, this method is not in accordance with the present invention since the grass silage fed in these experiments contained only a relatively small amount of chlorophyll. Moreover, chlorophyll can only be broken down in ruminants so that feeding chlorophyll to non-ruminants will have no significant effect on the PhA content.

In contrast to chlorophyll, phytol can be metabolised in non-ruminants. In the prior art, only laboratory animals have, however, been supplemented with phytol, primarily to elucidate the pathophysiology of Refsum's disease. In general, it was moreover noted that substantial morbidity as evidenced by growth retardation, weight loss and lethargy, already emerged from levels of supplementation of 1% of diet weight on and serious mortality rates were induced at levels of 5% [Steinberg 1966].

From the prior art it thus appears that phytol supplementation has such toxic effects on growth and health in laboratory animals that [Steinberg 1966] concluded, albeit within the context of the development of an animal model for Refsum's disease, that “the dosages of dietary phytol or phytanic acid needed to produce tissue accumulation of phytanic acid in normal animals are large and incompatible with growth and survival in the species tested.”

According to the invention it was found that, under standard zoo technical conditions, it appeared to be possible to achieve increased levels of PhA (or metabolites or derivatives thereof) in livestock products by supplementing the feed of livestock animals with phytol or other compounds forming or producing the above described PPAR/RXR heterodimer activator, more particularly, increased levels that have a beneficial effect on the health of the humans consuming the livestock products. This is quite surprising not only in view of the toxic effects of phytol but also in view of the fact that the branched nature of PhA seriously impedes the activity of several fatty acid enzymes that do not seem impacted as much by CLA. As indirect evidence, it was already noted that the presence of PhA in substantial proportion in the triglycerides and phospholipids was associated with the presence of phytenic acid (and not PhA) in the cholesterol esters of plasma [Steinberg 1966] but not with its deposition in quantity in a series of tissues. For example, PhA apparently inhibits the adipose tissue lipoprotein lipase, blocking its significant deposition in fat tissue. Also the mamary gland lipoprotein lipase discriminates against PhA, severely limiting the deposition of PhA in the milk, despite high plasma levels. Illustrative is also that the placental barrier is virtually impermeable to PhA [Lough 1977].

Consequently, one cannot extrapolate the deposition rate of PhA in the egg, for example, nor in the skeletal muscle of the growing organism. Granted, the deposition of PhA in the heart of grass fed steers was significant [Lough 1977]. Indeed, as the heart muscle is constantly active, it has an excessive and continuous energy requirement in contrast to other muscle types. As most of the energy is provided by fatty acids, the heart muscle has a very high turn over rate of its fatty acids. Consequently, dietary changes are more readily reflected in the fatty acid profile of the heart muscle, even if a particular compound, like PhA, is far from being the preferred substrate. However, skeletal muscles have much lower energy requirements and their main energy source is glycogen, not fatty acids. Therefore, the turn over rate of fatty acids in skeletal muscle is manyfold lower than that for the heart muscle and their metabolic enzymes are under a substantially different tissue specific control and configuration.

In the method according to the invention, the human diet is supplemented with a PPAR/RXR heterodimer activator in order to achieve beneficial health effects. The PPAR/RXR dimer activator is an agonist of any of the PPARs alpha and gamma and/or of the RXR enabling to activate the PPAR/RXR dimer so that it may straddle the peroxisome proliferator response elements (PPRE) on the DNA to trigger the transcription of a particular set of genes. The PPAR/RXR heterodimer activator employed in the present invention is phytanic acid, a metabolite of phytanic acid, a derivative of phytanic acid or of said metabolite, or a combination thereof. The PPAR/RXR heterodimer activator is advantageously phytanic acid, pristanic acid, TMTD (4,8,12trimethyltridecanoic acid), a derivative of these acids or a combination thereof, the PPAR/RXR heterodimer activator being preferably phytanic acid and/or pristanic acid.

In the method according to the invention, the level of one or more of these PPAR/RXR heterodimer activators is increased in livestock products, in particular in skeletal meat, milk and/or eggs, produced by livestock animals in agri- or aquaculture. This is achieved by making the livestock animals ingest at least one product that comprises the PPAR/RXR heterodimer activator and/or a precursor thereof, which is metabolised by the livestock animals into the PPAR/RXR heterodimer activator. The product can be in the form of a feed or a feed supplement fed to the animals (either via the feed or via the drinking fluids). An important advantage of the method according to the invention is that, by feeding the product to livestock animals instead of directly to humans, the human food itself is rendered more healthy but with at least one order of magnitude lower risk of overload, overdoses or adverse effects for the consumers.

When the livestock animals are ruminants, chlorofyll can be given as precursor of the PPAR/RXR heterodimer activator. This chlorophyll is preferably contained in a chlorophyll rich product containing at least 0.25% by dry weight, preferably of at least 0.50% by dry weight and more preferably of at least 0.75% by dry weight chlorophyll. Examples of such chlorophyll rich products are chlorophyll paste, Chlorella powder, dried blue green algae, Spirulina/Chlorella powder or tablets and Spirulina. Chlorophyll given in a less concentrated form contributes however also to the accumulation of the PPAR/RXR heterodimer activator. Consequently, grass, grass silage, alfalfa (which contains more chlorophyll than grass) and other natural feeds can be given, in combination with a product which has a higher content of the PPAR/RXR heterodimer activator and/or the precursor thereof in order to achieve the minimum amounts required by the invention.

Non-ruminants can be given metabolites of chlorophyll, i.e. first of all, phytol, which further metabolises into phytenic acid, phytanic acid, pristanic acid and TMTD. In view of the cost for producing it on an industrial scale, phytol is the preferred product to be given to the livestock animals in the present economic conditions. The other compounds are more expensive to produce per PPAR/RXR heterodimer activator equivalent, but can also be used in the method according to the invention. Possibly, use can be made of living organisms containing a relatively high level of these compounds, for feeding the livestock. On the other hand, chlorophyll can also be given to non-ruminants together with chemical or biological agents that are active to dissociate the phytyl chain from its chlorophyll parent molecule.

Instead of administering the above compounds respectively in the alcohol and in the acid form, they can also be administered in the form of a salt, an ester or an amide since these compounds will be converted back to the alcohol or the acid form in the gastro-intestinal system.

More generally, different derivatives of the above compounds and metabolites of phytol can be used provided they act as PPAR/RXR heterodimer activator or provided they are a precusor of such an activator in the livestock animals. Such compounds can be selected from the group of compounds corresponding to the following formulas: CH₃—CR₁H—CH₂ CH₂—CH₂—CR₂H—CH₂—CH₂—CH₂—CR₃H—CH₂—CH₂—(CH₂)ₘ—R₄ and CH₃—CR₁H—CH₂—CH₂—CH₂—CR₂H—CH₂—CH₂—CH₂—CR₃H—R₅,

wherein:

each of R₁, R₂, R₃ and R₆ is either CH₃, C₂H₅ or C₃H₇;

m=0−2;

R₄=CH₂—CR₆═CH—CH₂OH (phytol);

CH₂—CR₆═CH—CHO (phytenal);

CH₂—CR₆═CH—COOH (phytenic acid);

CH₂—CR₆H—CH₂—COOH (phytanic acid);

CH₂—CR₆H—CHOH—COOH (2-hydroxyphytanic acid);

CH₂—CR₆H—CH₂—CH₂OH;

CH₂—CO—CH₂—COOH;

CH₂—CR₆H—COOH (pristanic acid);

CHOH₂—CR₆H—COOH (3-hydroxypristanic acid);

CH₂—CR₆H—CH₂—CH₂OH;

CH₂—CR₆H—CHO (pristanal);

CH═CR₆—COOH (2, 3 pristenic acid);

CO—CR₆H—COOH (3 keto pristanic acid);

CH₂—CHOH—CH₂OH;

CH₂—CO—COOH;

CH₂—COOH;

CH₂—CHO;

CH₂—CH₂OH;

CHOH—CH₂OH;

CH₂—O—CHO;

COOH (4,8,12-TMTD); or

CHO and

R₅═CH₂—COOH or

COOH,

or which are a salt, an ester or an amide thereof, in particular chlorophyll, prophyrin, and phospholipid and di- or triacylglyceryl esters. The names between brackets are the names of the respective compounds when m=0 and R₁, R₂, R₃ and optionally R₆ is CH₃.

In the method according to the invention the product comprising the PPAR/RXR heterodimer activator or the precursor thereof is given in a predetermined minimum amount and for a period of time such that the PPAR/RXR heterodimer activator accumulates in the livestock animal and an increased level is obtained in the livestock product. The minimum amount of activator to be given over a period of at least three days is expressed as a ratio of the amount feed dry matter ingested by the livestock animals during that period. In order to exclude any effect of the molecular weight of the activator or precursor and in order to exclude the effect of any difference in the number of functional activator groups in the precursor, the amount of activator is further expressed in milli-equivalents, more particularly in PPAR/RXR heterodimer activator milli-equivalents. One millimole of phytol, i.e. 294 mg of phytol, thus corresponds to one meq phytol. For example, when a precursor is used such as a di- or a triglyceride containing two or three phytanate groups, one mole corresponds to two or respectively three equivalents of the di- or the triglyceride.

When the livestock animals eat a total amount of F kg feed dry weight over said period of time, they should be made to ingest an amount of the product which contains at least 5×F meq, preferably at least 10×F meq, and more preferably at least 15×F meq of said PPAR/RXR heterodimer activator and/or precursor thereof. When different activators and/or precursors are used, the sum of the respective amounts of these compounds should be greater than the minimum amount, provided the different compounds are available for the livestock animal, i.e. provided the compounds can be taken up and, if necessary, converted into the PPAR/RXR dimer activator. When phytol is used, the above amounts correspond to about 0.15, 0.3 and 0.45% of dry diet weight, respectively.

During said period of time, the product can be given one or several times. Preferably, the product is given at least once a day and is more preferably given with the feed of the livestock animals. The product can be given separately from the feed but preferably it is mixed therewith. The present invention also provides a feed for livestock animals which is composed to contain at least 5 meq/kg feed dry weight, preferably at least 10 meq/kg feed dry weight, and more preferably at least 15 meq/kg feed dry weight of the PPAR/RXR heterodimer activator and/or precursor thereof, preferably phytol. This feed can either be manufactured in advance or the farmer can also prepare it by mixing a product containing the PPAR/RXR heterodimer activator and/or precursor thereof with other feed constituents. Optionally, the product can also be administered via the drinking fluids.

The product is preferably given in said amounts over more than one period of at least three days or over one or more longer periods, more particularly over at least one period of at least one week, more preferably over at least one period of at least two weeks so that it further accumulates in the livestock animal. When the livestock animals are slaughtered to produce the livestock product, in particular skeletal meat, the livestock animals are made to ingest the product preferably for at least three days during the last week before slaughtering. Of course, the product can already been given before the last week and also during the entire last week. During the last days, it can moreover be given in an increased amount in order to achieve a maximum level in the livestock product upon slaughtering.

Compared to the therapeutic amounts of phytol and phytanic acid, the amounts given in accordance with the present invention are relatively high, and are, in particular, considerably higher than the amounts which can be achieved by feeding grass or even alfalfa to ruminants. For a pig of 80 kg having a daily dry feed intake of 2 kg, the amounts of 5×F meq, 10×F meq and 15×F meq correspond to 37 mg, 74 mg and 111 mg/kg body weight, respectively. For a chicken of 2 kg having a daily dry feed intake of 0.1 kg, these amounts correspond even to 74 mg, 148 mg and 222 mg/kg body weight, respectively. In order to achieve an even higher accumulation of the PPAR/RXR heterodimer activator, the livestock animals can be made to ingest, over said period of time, at least 25×F meq, preferably at least 35×F meq, more preferably at least 50×F meq and most preferably at least 65×F meq of the PPAR/RXR heterodimer activater and/or precursor thereof. When phytol is used, these amounts correspond to about 0.75, 1.0, 1.5 and 2.0% of dry diet weight, respectively. Preferably, the livestock animals are made to ingest, over said period of time, less than 175×F meq, and more preferably less than 125×F meq of the PPAR/RXR heterodimer activater and/or precursor thereof.

By means of the method according to the invention, livestock products can be produced having certain minimum levels of the PPAR/RXR heterodimer activator, in particular of phytanic acid, pristanic acid and/or TMTD, by giving the products containing this or these activators and/or precursors thereof in a sufficiently large amount and for a sufficient long period of time.

In the present specification the level of the PPAR/RXR heterodimer activator is expressed as a percentage of total FAME fatty acids. These total FAME fatty acids comprise those fatty acids with a linear chain of at least 12 carbons and are measured by the so-called FAME technique, which is well known for the skilled person and wherein, first, fatty acid methyl esters are prepared which are, subsequently, analysed via gas chromatography. The FAME procedure used for determining the results obtained by the present invention was as follows. Lipids were extracted from the samples using a dissolving solution that is specific to each sample type. Nonadecanoic acid (19:0) was added as an internal standard. The two-step methylation procedure consisted of using a basic reagent NaOH/methanol followed by an acid reagent HCI/methanol. The fatty acid methyl esters (FAME) were analyzed by GC (HP 6890, Hewlett-Packard, Brussels, Belgium) using a CP-Sil88 column for FAME (100 m×250 μm×0.25 μm) (Chrompack, Middelburg, The Netherlands). The GC conditions were as adapted to each sample type. Peaks were identified by comparison of retention times with those of the corresponding standards (Sigma, Botnew, Belgium; Nu-Chek-Prep, Elysian, M N). Identification of the peaks included fatty acids between 12:0 and 22:6 and 5 different CLA isomers and phytanic acid and pristanic acid.

The product can be given to non-ruminant mammals and to poultry (broilers) so that a level of said PPAR/RXR heterodimer activator of at least 0.2%, preferably of at least 0.5% and more preferably of at least 1.0% of total FAME fatty acids is achieved in said livestock product, in particular, in skeletal meat of the livestock animals. The non-ruminant mammals are preferably non-rodents since it has been found that non-rodents, generally, do not show the peroxisome proliferation upon activation of the PPAR/RXR heterodimer that is typical in laboratory mice.

When the product is given to poultry (layers) producing eggs as the livestock product, the product is preferably given so that a level of said PPAR/RXR heterodimer activator of at least 1%, preferably of at least 3% and more preferably of at least 5% of total FAME fatty acid is achieved in egg yolk of said eggs.

When the product is given to ruminants producing skeletal meat as the livestock product, the product is preferably given so that a level of said PPAR/RXR heterodimer activator of at least 0.7%, preferably of at least 0.9% and more preferably of at least 1.0% of total FAME fatty acid is achieved in skeletal meat of the livestock animals.

When the product is given to ruminants producing milk as the livestock product, the product is preferably given so that a level of said PPAR/RXR heterodimer activator higher than 0.75%, preferably higher than 1.0% and more preferably higher than 1.5% of total FAME fatty acid is achieved in milk of the livestock animals.

When the product is given to aquatic animals such as the aquatic animals defined in the main group 4 “Fish and fish products” of the Europcode 2 version of Apr. 8, 1999, which are incorporated herein by way of reference, used to produce the livestock product in aquaculture, the product is preferably given so that a level of said PPAR/RXR heterodimer activator of at least 0.7%, preferably of at least 0.9% and more preferably of at least 1.0% of total FAME fatty acid is achieved in the livestock product.

The method according to the invention cannot only be applied to accumulate the PPAR/RXR heterodimer activator in livestock products but it also enables to improve the carcass quality of livestock animals. In particular for pigs, it has been observed that, from a group of pigs that were given phytol, a number of pigs did no longer gain weight but exhibited a carcass configured towards more lean mass.

Experiments have also shown that, for some kinds of livestock animals, the supplementation of the feed with the PPAR/RXR heterodimer activator or the precursor thereof has, within a population of the same livestock animals, a different effect on a certain parameter so that the population can be split up into two groups. For chicken (broilers) it has for example be observed that the feeding of phytol causes in one group of chicken a greater accumulation of phytanic acid than in the other group. A selection can thus be made for chicken showing the largest accumulation of PhA. For pigs, it has on the other hand been observed that, within one population, there were two groups, namely one group which fails to gain weight when being made to ingest phytol whilst another group gained weight to a comparable extend as a control group. When, as explained hereabove, an improved carcass quality is the production goal, one should continue the phytol administering to the group of pigs that do not gain weight whilst when only an accumulation of the PPAR/RXR heterodimer activator is the production goal, one should continue with the group of pigs which gained weight.

### EXAMPLE 1

### Broilers

ROSS 308 broiler chicks were raised, lege artis, on an ad libitum diet, containing phytol at 2% by weight of feed that, characteristically, contains about 10% of humidity. The chicks consumed an average of about 0.1 kg dry weight of the feed per day. The phytol in the diet replaced 2% of the soybean oil included in feeds formulated based on the INVE Nutritional Requirement standard formula for a grower feed (formula 120). Please refer to the following tables for the feed formula and for its chemical composition.

Broiler feed composition

| | | Composition |
|---|---|---|
| | | (%) |
| 300 | Corn | 26.00 |
| 800 | Wheat | 28.70 |
| 1402 | Fullfat soybeans, toated | 17.00 |
| 1424 | Soybean meal 48 + 2 | 17.00 |
| 2815 | Patatoprotein | 2.20 |
| 4200 | Soybean oil | 2.00 |
| 4370 | INVE fat | 3.70 |
| 5100 | Monocalciumphospate | 0.97 |
| 5150 | Limestone | 0.87 |
| 5170 | Salt | 0.28 |
| 5173 | Sodiumbicarbonate | 0.27 |
| 5300 | L-lysine | 0.17 |
| 5301 | DL-Methionin | 0.24 |
| 5303 | L-threonine | 0.05 |
| 6511 | Sacox 12%* | 0.05 |
| 84928 | INVE Broiler 0.5% | 0.50 |
| | Sum | 100.00 |

Chemical composition of broiler feed

| | Composition |
|---|---|
| | (g/kg) |
| Dry matter | 889 |
| Crude ash | 81 |
| Crude protein | 212 |
| Fat | 106 |
| Starch | 344 |
| Crude fibres | 31 |
| Ca | 8.0 |
| Total P | 5.4 |
| Av. P | 4.0 |
| Ca/Av. P | 2.0 |
| Dig lysine poultry | 11.0 |
| Dig met/dig lys | 0.47 |
| Dig met + cyst/dig lys | 0.73 |
| Dig thr/dig lys | 0.65 |
| Dig try/dig lys | 0.21 |
| MEn broiler (kCal) | 3021 |
| MEn broiler (kJ) | 12.6 |
| MEn poultry (kCal) | 3259 |
| MEn poultry (kJ) | 13.6 |

The animals were slaughtered after 42 days and their tissues sampled for analysis.

During the feeding trial, there was no difference in mortality or morbidity when compared with a group that received the standard broiler feed without the phytol supplement. It was observed that the final body weight (2122 g vs. 1842 g), the feed conversion rate (1.818 vs. 2.120) and the ratio breast weight/total weight (15.9% vs. 14.4%) were roughly one tenth less advantageous under the phytol supplementation diet, but still well within acceptable zoo technical ranges.

The fatty acid analysis of breast meat showed that PhA reached an average level of 2.6% of total fatty acids. Noteworthy was also a serious drop in PUFA (polyunsaturated fatty acid) content that is explained by the lack of 2% of soybean oil in the phytol supplemented diet.

Closer inspection of the results revealed that the broilers in the treatment group could be classified neatly into two subgroups according to the content of PhA in the breast meat, with values of one subgroup clustered around 1.9% and the values of the other subgroup clustered around 3.6%, almost double. This illustrates clearly the emergence of a heretofore silent phenotype under conditions that put the metabolic pathway of PhA under heavier loads. If the initial weight gain is correlated with this final PhA deposition reate, a selection is possible by phenotype after a short feeding trial to continue the finishing with those individual animals with the most effective phenotype.

### EXAMPLE 2

### Layers

48 week old ISABROWN layers were kept, lege artis, and fed ad libitum a diet containing phytol at 2% by feed weight. The layers consumed on average about 0.1 kg dry weight of the feed a day. The phytol replaced 2% of soybean oil included an INVE layer formulation with the following feed composition.

Layer feed composition

| | | Composition |
|---|---|---|
| | | (%) |
| 300 | Corn | 45.50 |
| 800 | Wheat | 20.00 |
| 1402 | Fullfat soybeans, toasted | 22.00 |
| 4200 | Soybean oil | 2.00 |
| 5100 | Monocalciumphospate | 0.77 |
| 5150 | Limestone | 2.20 |
| 5152 | Limestone SEM white | 6.50 |
| 5170 | Salt | 0.23 |
| 5173 | Sodiumbicarbonate | 0.18 |
| 5301 | DL-Methionin | 0.12 |
| 84928 | INVE Broiler 0.5% | 0.50 |
| | Sum | 100.00 |

There was no difference in mortality nor morbidity in comparison with a group fed a standard layer diet without phytol supplementation. Although the daily egg mass was lower with the supplemented diet, the feed conversion rate remained zoo technically within acceptable ranges. This is shown in this table below:

| | Laying | | Daily | | |
|---|---|---|---|---|---|
| | rate | Egg weight | egg mass | ADFI | |
| | (%) | (g/a/d) | (g/a/d) | (g/a/d) | FCR |
| control | 90.3 | 65.6 | 59.2 | 112.3 | 1.9 |
| 2% phytol fed | 83.0 | 62.8 | 52.1 | 103.8 | 2.0 |

The quality of the eggs with respect to standard parameters for shell quality and color of the yolk did not change significantly except for a less reddish coloring of the yolk in the supplemented group. The fatty acid analysis of the egg yolk revealed that supplementing the diet with 2% by weight phytol resulted in a deposit of 11.5% of total FAME fatty acids of the branched chain fatty acids PhA, mainly, and a trace of PrA. Surprisingly, it appeared that the PhA displaced almost exclusively the mono unsaturated fatty acids.

### EXAMPLE 3

### Pork

Hybrid boars weighing in at about 80 kg were kept lege artis and fed a standard finishing granulated feed sprayed on with phytol at a level of 2% of feed weight. The feed was formulated and produced by Schatteman in Wetteren, Belgium and contained on average about 7% of humidity. On spraying, the feed readily absorbed this oily substance. The boars were fed the phytol supplemented granulated feed ad libitum and consumed on average about 1.8 kg of this feed per day. After one month, the boars were slaughtered. Tissue samples were taken and the carcass quality assessed. The carcasses were further butchered in the usual fashion to chops, loins, sausages and the like and the meat quality of the prime cuts was assessed.

During the finishing period no difference in feeding behavior or level of activity was observed between the boars fed the usual diet and those fed the phytol enriched feed. Also, no animals got sick or died during the entire period. At slaughter, the boars in the intervention group could be divided into two groups according to their slaughter weight: a group which thrived and gained weight comparable to boars which had received the standard diet (weight gain 13.1 vs 11.2 kg) and another group which thrived but failed to gain weight. We presume that, as is usual in pig rearing, genetic variability accounts for these differences. Obviously, in practice, one could introduce a feed trial for a week and continue on with the supplemented diet only with those animals that showed already a significant weight gain or select those prone to carcass fat to lean mass redistribution to increase the carcass quality.

| | initial | final | | | quality | chinese | moisture |
|---|---|---|---|---|---|---|---|
| | weight | weight | % meat | type-index | class | color | loss |
| control group | 80000 | 91500 | 60.88 | 1.79 | A1 | 2.50 | 0.045 |
| | 81000 | 95000 | 57.61 | 2.25 | A2 | 3.00 | 0.043 |
| | 88000 | 99500 | 51.32 | 2.54 | B2 | 2.50 | 0.085 |
| | 79000 | 94500 | 59.42 | 2.07 | A1 | 2.00 | 0.067 |
| 2% phytol group | 80000 | 93000 | 57.99 | 2.42 | B2 | 2.50 | 0.067 |
| | 82000 | 91500 | 56.38 | 1.93 | A2 | 2.50 | 0.065 |
| | 80000 | 77500 | 58.89 | 2.3 | A1 | 3.50 | 0.031 |
| | 87500 | 83000 | 60.62 | 1.83 | A1 | 2.50 | 0.039 |
| average control | 82000 | 95125 | 57.31 | 2.16 | | 2.50 | 0.060 |
| average 2% phytol | 82375 | 86250 | 58.47 | 2.12 | | 2.75 | 0.050 |
| average gainers | 81000 | 92250 | 57.19 | 2.18 | | 2.50 | 0.066 |
| average no-gainers | 83750 | 80250 | 59.76 | 2.07 | | 3.00 | 0.035 |

With respect to the quality of the carcasses and the meat, no significant differences were found with those fed the standard diet. The quality was assessed objectively using the following parameters: % meat on the carcass, type-index, meat class, meat moisture, meat color, meat temperature and meat pH change. It was remarkable that the group that failed to gain weight produced top quality, lean and good muscled carcasses (quality class A1).

| | | | | | | | | | average | average |
|---|---|---|---|---|---|---|---|---|---|---|
| color | control group | | | | 2% phytol group | | | | control | 2% phytol |
| L (avg) | 53.83 | 51.65 | 54.60 | 57.65 | 54.05 | 55.95 | 49.26 | 52.03 | 54.43 | 52.82 |
| a (avg) | 6.40 | 8.08 | 7.65 | 5.78 | 7.92 | 6.21 | 8.10 | 7.57 | 6.98 | 7.45 |
| b (avg) | 15.07 | 15.40 | 15.16 | 15.08 | 15.56 | 14.97 | 13.84 | 15.09 | 15.18 | 14.87 |
| 40 minutes | | | | | | | | | | |
| pH L carré | 5.94 | 6.14 | 6.02 | 6.00 | 5.83 | 5.88 | 5.95 | 6.01 | 6.03 | 5.92 |
| pH R carré | 6.14 | 6.13 | 5.99 | 5.84 | 5.70 | 5.84 | 6.01 | 5.90 | 8.03 | 5.86 |
| pH L ham | 6.17 | 5.87 | 6.19 | 5.98 | 5.95 | 6.40 | 5.93 | 6.09 | 6.05 | 6.09 |
| pH H carré | 6.29 | 5.90 | 6.13 | 6.01 | 5.72 | 6.51 | 5.90 | 6.98 | 6.08 | 6.28 |
| T L carré (° C.) | 37.80 | 38.90 | 40.70 | 37.80 | 39.60 | 40.20 | 38.70 | 39.00 | 38.83 | 39.38 |
| T T carré (° C.) | 37.80 | 39.70 | 40.60 | 37.60 | 38.10 | 39.80 | 39.00 | 39.40 | 38.93 | 39.08 |
| 24 hours | | | | | | | | | | |
| pH L carré | 5.30 | 5.27 | 5.16 | 5.21 | 5.18 | 5.19 | 5.33 | 5.18 | 5.24 | 5.22 |
| pH R carré | 5.29 | 5.21 | 5.28 | 5.19 | 5.16 | 5.18 | 5.29 | 5.31 | 5.24 | 5.24 |
| pH L ham | 5.29 | 5.31 | 5.33 | 5.25 | 5.35 | 5.29 | 5.32 | 5.29 | 5.30 | 5.31 |
| pH R carré | 5.33 | 5.37 | 5.41 | 5.28 | 5.29 | 5.29 | 5.33 | 5.28 | 5.35 | 5.30 |

With respect to the further processing of the pork, there were no noticeable differences in handling and transforming the meat.

With respect to the content of PhA and PrA in the pork meat, levels averaging 2.3% of total FAME fatty acids were found. It was also remarkable that the inclusion of these branched chain fatty acids did not produce a significant shift in the remainder of the fatty acid profile like towards less unsaturated fatty acids as is commonly found in CLA supplementation experiments.

### EXAMPLE 4

### Shrimp

Tiger shrimp (Penaeus Monodon), weighing in at 0.7 g a piece, were kept lege artis and fed a diet containing phytol at 2% of pellet diet weight and this during 4 weeks. The feeds were extruded using a standard shrimp grow out recipe as developed by INVE Technologies nv, Dendermonde, Belgium, where the phytol replaced 2% of soybean oil. The shrimp were allocated 20 a piece in triplicate tanks of 500 liters. After a week of acclimatization the shrimp were fed at a daily rate of about 15% of biomass weight.

| | | |
|---|---|---|
| Wheat Flour | 43.319 | 43.319 |
| Fish Meal Standard 999 | 35.000 | 35.000 |
| Defatted Soya Flour 50 | 9.610 | 9.610 |
| Shrimphead Meal | 4.000 | 4.000 |
| Wheat Gluten | 2.000 | 2.000 |
| Soya Oil | 2.000 | 0.000 |
| Phytol | 0.000 | 2.000 |
| Squid meal | 1.000 | 1.000 |
| Brewers Yeast | 0.750 | 0.750 |
| Lecithin | 0.679 | 0.679 |
| Fish Oil | 0.642 | 0.642 |
| INVE Premix | 1.000 | 1.000 |
| | 100.0 | 100.0 |

Formula shrimp finishing

| | | |
|---|---|---|
| Moisture | 10.20 | 7.37 |
| Crude Protein | 38.00 | 39.53 |
| Crude Fibre | 1.20 | 1.23 |
| Crude Ash | 8.02 | 8.35 |
| Crude Fat after Hydrolysis | 8.80 | 8.37 |

Proximate Analysis (% in diet)

At the end of the feeding trials, there was no difference in survival rate compared with a similar triplicate group fed the standard diet without phytol supplementation. The delay on growth in the supplemented diet group was marked but still satisfactory from a zoo technical point of view (2.05 g vs. 3.3 g). During the first two weeks of feeding, the consumption of feeds in both groups was similar, although the growth rate differed already at about the same proportion. During the last two weeks however, the supplemented diet group consumed considerably less feed (53.7 vs 66.5 g), thus partially correcting an initially less attractive feed conversion ratio. Moreover, the shrimps used in this example were quite young, for more adult shrimp, the delay on growth is expected to be even smaller.

Fatty acid analysis revealed that during that feeding period the shrimp tissue had accumulated an average of 5.3% of TFA (total fatty acids) of PhA. Also, the total fat content dropped with about a fifth, a potential marketing advantage.

### CLA BIBLIOGRAPHY

Adlof R O, Duval S, Emken E A. Biosynthesis of conjugated linoleic acid in humans. Lipids. 2000 February;35(2):131-5.
Basu S, Smedman A, Vessby B. Conjugated linoleic acid induces lipid peroxidation in humans. FEBS Lett. 2000 Feb. 18;468(1):33-6.
Belury M A. Inhibition of carcinogenesis by conjugated linoleic acid: potential mechanisms of action. J Nutr. 2002 October;132(10):2995-8.
Chamruspollert M, Sell J L. Transfer of dietary conjugated linoleic acid to egg yolks of chickens. Poult Sci. 1999 August;78(8):1138-50.
Chilliard Y, Ferlay A, Doreau M. Effect of different types of forages, animal fat or marine oils in cow's diet on milk fat secretion and composition, especially conjugated linoleic acid (CLA) and polyunsaturated fatty acids. Livest. Prod. Sci. 2000 July;70(1-2):31-48.
Choi B D, Kang S J, Ha Y-L, Ackman R G. Accumulation of conjugated linoleic acid (CLA) in tissues of fish fed diets containing various levels of CLA in “Quality attributes of muscle foods” Xiong Y L, Ho C T, Shahidi F. eds. 1999 Kluwer academic/Plenum publisher, NY 61-71
Chuang L T, Leonard A E, Liu J W, Mukerji P, Bray T M, Huang Y S. Inhibitory effect of conjugated linoleic acid on linoleic acid elongation in transformed yeast with human elongase. Lipids. 2001 October;36(10):1099-103.
Decker E A. The role of phenolics, conjugated linoleic acid, carnosine, and pyrroloquinoline quinone as nonessential dietary antioxidants. Nutr Rev. 1995 March;53(3):49-58. Review.
Fritsche J, Rickert R, Steinhart H. 1999. Formation, contents, and estimation of daily intake of conjugated linoleic acid isomers and trans-fatty acids in foods. In Yuraweez M P, Mossobo M M, Kramer J K G, Pariza M W, Nelson G J, eds. 1999. Advances in Conjugated Linoleic Acid Research, Vol. 1. Champaign, Ill.: AOCS Press, pp. 378-96
Fukuda S, Ninomiya N, Asanuma N, Hino T. Production of Conjugated Linoleic Acid by Intestinal Bacteria in Dogs and Cats. J Vet Med Sci. 2002;64(11):987-992.
Ha Y L, Grimm N K, Pariza M W 1987. Anticarcinogens from fried ground beef: heat-altered derivatives of linoleic acid. Carcinogenesis 8,1881-1887.
Hamalainen T I, Sundberg S, Hase T, Hopia A. Stereochemistry of the hydroperoxides formed during autoxidation of CLA methyl ester in the presence of alpha-tocopherol. Lipids. 2002 June;37(6):533-40.
Houseknecht K L, Vanden Heuvel J P, Moya-Camarena S Y, Portocarrero C P, Peck L W, et al. 1998. Dietary conjugated linoleic acid normalizes impaired glucose tolerance in the Zucker diabetic atty fa/fa rat. Biochem. Biophys. Res. Commun. 244:678-82
Joo S T, Lee J I, Ha Y L, Park G B. Effects of dietary conjugated linoleic acid on fatty acid composition, lipid oxidation, color, and water-holding capacity of pork loin. J Anim Sci. 2002 January;80(1):108-12.
Lee K N, Pariza M W, Ntambi J M. Conjugated linoleic acid decreases hepatic stearoyl-CoA desaturase mRNA expression. Biochem Biophys Res Commun. 1998 Jul. 30;248(3):817-21.
U Y, Seifert M F, Ney D M, Grahn M, Grant A L, et al. 1999. Dietary conjugated linoleic acids alter serum IGF-I and IGF binding protein concentrations and reduce bone formation in rats fed n-6 or n-3 fatty acids. J. Bone Miner. Res. 14:1153-62
Loor J J, Lin X, Herbein J H. Dietary trans-vaccenic acid (trans11-18:1) increases concentration of cis9,transll-conjugated linoleic acid (rumenic acid) in tissues of lactating mice and suckling pups. Reprod Nutr Dev. 2002 Mar-Apr;42(2):85-99.

Moya-Camarena S Y, Van den Heuvel J P, Belury M A. Conjugated linoleic acid activates peroxisome proliferator-activated receptor alpha and beta subtypes but does not induce hepatic peroxisome proliferation in Sprague-Dawley rats. Biochim Biophys Acta. 1999 Jan. 4;1436(3):331-42.

Offer N W, Dixon J, Speake B K, 1998. Effect of dietary fat supplements on levels of trans acids and CLA in bovine milk. CLA What(s going on. European concerted action No. 1, p 4.

Ostrowska E, Muralitharan M, Cross R F, Bauman D E, Dunshea F R. Dietary conjugated linoleic acids increase lean tissue and decrease fat deposition in growing pigs. J Nutr. 1999 November;129(11):2037-42.

Pariza M W, Park Y, Cook M E. The biologically active isomers of conjugated linoleic acid. Prog Lipid Res. 2001 July;40(4):283-98. Review.

Park Y, Storkson J M, Ntambi J M, Cook M E, Sih C J, Pariza M W. Inhibition of hepatic stearoyl-CoA desaturase activity by trans-10, cis-12 conjugated linoleic acid and its derivatives. Biochim Biophys Acta. 2000 Jul 19;1486(2-3):285-92.

Park Y, McGuire M K, Behr R, McGuire M A, Evans M A, Schultz T D. 1999. High-fat dairy product consumption ncreases19c, 11t-18:2 (rumenic acid) and total lipid concentrations of human milk. Lipids 34:543-49

Raes K, Huyghebaert G, De Smet S, Nollet L, Arnouts S, Demeyer D. The deposition of conjugated linoleic acids in eggs of laying hens fed diets varying in fat level and fatty acid profile. J Nutr. 2002 February;132(2):182-9.

Ramsay T G, Evock-Clover C M, Steele N C, Azain M J. Dietary conjugated linoleic acid alters fatty acid composition of pig skeletal muscle and fat. J Anim Sci. 2001 August;79(8):2152-61.

Reaney M J T, Liu Y D, Westcott N D. Commercial production of conjugated linoleic acid in “Advances in Conjugated Linoleic Acid Research” Yurawecz M P, Mossobo M M, Kramer J K G, Pariza M W, Nelson G J, eds. 1999, Vol. 1. Champaign, Ill.: AOCS Press, pp. 39-54
Riserus U, Basu S, Jovinge S, Fredrikson G N, Arnlov J, Vessby B. Supplementation with conjugated linoleic acid causes isomer-dependent oxidative stress and elevated C-reactive protein: a potential link to fatty acid-induced insulin resistance. Circulation. 2002 Oct. 8;106(15):1925-9.
Ryder J W, Portocarrero C P, Song X M, Cui L, Yu M, Combatsiaris T, Galuska D, Bauman D E, Barbano D M, Charron M J, Zierath J R, Houseknecht K L. Isomer-specific antidiabetic properties of conjugated linoleic acid. Improved glucose tolerance, skeletal muscle insulin action, and UCP-2 gene expression. Diabetes. 2001 May;50(5):1149-57.
Saebo A Commercial production of CLA in Conjugated linoleic acid—emerging nutrient of the 21st century. Society of Chemical industry seminar on 15 Mar. 2001.
Schafer K, Manner K, Sagredos A, Eder K, Simon O. Incorporation of dietary linoleic and conjugated linoleic acids and related effects on eggs of laying hens. Lipids. 2001 November;36(11):1217-22.
Smedman A, Vessby B. 2001. Conjugated linoleic acid supplementation in humans-metabolic effects. J. Nutr. 36:773-81
Sugano M, Tsujita A, Yamasaki M, Noguchi M, Yamada K. 1998. Conjugated linoleic acid modulates tissue levels of chemical mediators and immunoglobulins in rats. Lipids 33:521-27
Szymczyk B, Pisulewski P M, Szczurek W, Hanczakowski P. Effects of conjugated linoleic acid on growth performance, feed conversion efficiency, and subsequent carcass quality in broiler chickens. Br J Nutr. 2001 April;85(4):465-73.
Thiel-Cooper R L, Parrish F C Jr, Sparks J C, Wiegand B R, Ewan R C. Conjugated linoleic acid changes swine performance and carcass composition. J Anim Sci. 2001 July;79(7):1821-8.
Twibell R G, Watkins B A, Rogers L, Brown P B. Effects of dietary conjugated linoleic acids on hepatic and muscle lipids in hybrid striped bass. Lipids. 2000 February;35(2):155-61.
Twibell R G, Watkins B A, Brown P B. Dietary conjugated linoleic acids and lipid source alter fatty acid composition of juvenile yellow perch, Perca flavescens. J Nutr. 2001 September;131(9):2322-8.
Urquhart P, Parkin S M, Rogers J S, Bosley J A, Nicolaou A. The effect of conjugated linoleic acid on arachidonic acid metabolism and eicosanoid production in human saphenous vein endothelial cells. Biochim Biophys Acta. 2002 Feb. 28;1580(2-3):150-60.
Wilson T A, Nicolosi R J, Chrysam M, Kritchevsky D. 2000. Conjugated linoleic acid reduces early aortic atherosclerosis greater than linoleic acid in hypercholesterolemic hamsters. Nutr. Res. 20:1795-805
Yu Y, Correll P H, Vanden Heuvel J P. Conjugated linoleic acid decreases production of pro-inflammatory products in macrophages: evidence for a PPAR gamma-dependent mechanism. Biochim Biophys Acta. 2002 Apr 15;1581 (3):89-99.
Yurawecz M P, Mossobo M M, Kramer J K G, Pariza M W, Nelson G J, eds. 1999. Advances in Conjugated Linoleic Acid Research, Vol. 1. Champaign, Ill.: AOCS Press

### PhA BIBLIOGRAPHY

Eldjarn L, Jellum E, Aas M, Try K, Stokke O. Synthesis of 2,6,10,14-tetramethylpentadecanoic acid (pristanic acid). Acta Chem Scand. 1966;20(8):2313-4.
Ellinghaus P, Wolfrum C, Assmann G, Spener F, Seedorf U. Phytanic acid activates the peroxisome proliferator-activated receptor alpha (PPARalpha) in sterol carrier protein 2-/ sterol carrier protein x-deficient mice. J Biol. Chem. 1999 Jan. 29;274(5):2766-72.
Kitareewan S, Burka L T, Tomer K B, Parker C E, Deterding L J, Stevens R D, Forman B M, Mais D E, Heyman R A, McMorris T, Weinberger C. Phytol metabolites are circulating dietary factors that activate the nuclear receptor RXR. Mol Biol Cell. 1996 August;7(8):1153-66. PMID: 8856661
Lenhard J M. PPAR gamma/RXR as a molecular target for diabetes. Receptors Channels. 2001;7(4):249-58. Review. PMID: 11697231
Lough A K. The PHA content of the lipids of bovine tissues and milk. Lipids. 1977 January;12 (1):115-9. PMID: 834118
Masters-Thomas A, Bailes J, Billimoria J D, Clemens M E, Gibberd F B, Page N G. Heredopathia atactica polyneuritiformis (Refsum's disease): 2. Estimation of PHA in foods. J Hum Nutr. 1980 August;34(4):251-4. PMID: 6157717
McCarty M F. The chlorophyll metabolite PHA is a natural rexinoid—potential for treatment and prevention of diabetes. Med Hypotheses. 2001 February;56(2):217-9. PMID: 11425290
Mukherjee R, Davies P J, Crombie D L, Bischoff E D, Cesario R M, Jow L, Hamann L G, Boehm M F, Mondon C E, Nadzan A M, Paterniti J R Jr, Heyman R A. Sensitization of diabetic and obese mice to insulin by retinoid X receptor agonists. Nature. 1997 Mar. 27;386(6623):407-10.

Pineda Torra I, Gervois P, Staels B. Peroxisome proliferator-activated receptor alpha in metabolic disease, inflammation, atherosclerosis and aging. Curr Opin Lipidol. 1999 April;10(2):151-9. PMID: 10327283
Roberts-Thomson S J. Peroxisome proliferator-activated receptors in tumorigenesis: targets of tumour promotion and treatment. Immunol Cell Biol. 2000 August;78(4):436-41. PMID: 1094787
Steinberg D, Avigan J, Mize C E, Baxter J H, Cammermeyer J, Fales H M, Highet P F. Effects of dietary phytol and PHA in animals. J Lipid Res. 1966 September;7(5):684-91. PMID: 4165840
Van den Branden C, Vamecq J, Wybo I, Roels F. Phytol and peroxisome proliferation. Pediatr Res. 1986 May;20(5):411-5
Verhoeven N M, Jakobs C. Human metabolism of PHA and pristanic acid. Prog Lipid Res. 2001 November;40(6):453-66. Review. PMID: 11591435

1. A non-therapeutic method for achieving an increased level of at least one PPAR/RXR heterodimer activator in a livestock product for human consumption, in particular in skeletal meat, milk and/or eggs, in which method livestock animals, used in agri- or aquaculture for producing the livestock product, are made to ingest at least one product comprising said PPAR/RXR heterodimer activator and/or a precursor thereof which is metabolised by the livestock animals into said PPAR/RXR heterodimer activator, over such a period of time and in such an amount that the PPAR/RXR heterodimer activator is accumulated in the livestock animal so that said increased PPAR/RXR heterodimer activator level is achieved in the livestock product, characterised in that said PPAR/RXR heterodimer activator is phytanic acid, a metabolite of phytanic acid, a derivative of phytanic acid or of said metabolite, or a combination thereof and, in order to accumulate the PPAR/RXR heterodimer activator in the livestock animal, a predetermined amount of said product is given to the livestock animals over at least one period of at least three days, during which the livestock animals ingest a total amount of F kg feed dry weight, which predetermined amount of said product contains at least 5×F meq, preferably at least 10×F meq, and more preferably at least 15×F meq of said PPAR/RXR heterodimer activator and/or precursor thereof.

2. A method according to claim 1, characterised in that said product comprises as said PPAR/RXR heterodimer activator or as said precursor thereof at least one compound selected from the group of compounds which correspond to the following formulas:
CH₃—CR₁H—CH₂—CH₂—CH₂—CR₂H—CH₂—CH₂—CH₂—CR₃H—CH₂—CH₂—(CH₂)ₘ—R₄ and CH₃—CR₁H—CH₂—CH₂—CH₂—CR₂H—CH₂—CH₂—CH₂—CR₃H—R₅,
wherein:
each of R₁, R₂, R₃ and R₆ is either CH₃, C₂H₅ or C₃H₇;
m=0−2;
R₄=CH₂—CR₆═CH—CH₂OH;
CH₂—CR₆═CH—CHO;
CH₂—CR₆═CH—CO₂OH;
CH₂—CR₆H—CH₂—COOH;
CH₂—CR₆H—CHOH—COOH;
CH₂—CR₆H—CH₂—CH₂OH;
CH₂—CO—CH₂—COOH;
CH₂—CR₆H—COOH;
CHOH₂—CR₆H—COOH;
CH₂—CR₆H—CH₂—CH₂OH;
CH₂—CR₆H—CHO;
CH═CR₆—COOH;
CO—CR₆H—COOH;
CH₂—CHOH—CH₂OH;
CH₂—CO—COOH;
CH₂—COOH;
CH₂—CHO;
CH₂—CH₂OH;
CHOH—CH₂OH;
CH₂—O—CHO;
COOH; or
CHO and
R₅=CH₂—COOH or
COOH,
or which are a salt, an ester or an amide thereof, in particular chlorophyll, porphyrin, and phospholipid and di- or triacylglyceryl esters.

3. A method according to claim 2, characterised in that said product comprises as said PPAR/RXR heterodimer activator or as said precursor thereof at least one compound selected from the group of compounds which correspond to the following formulas:
CH₃—CR₁H—CH₂—CH₂—CH₂—CR₂H—CH₂—CH₂—CH₂—CR₃H—CH₂—CH₂—(CH₂)ₘ—R₄
wherein:
R₁, R₂, R₃ and R₆=CH₃;
m=0; and
R₄=CH₂—CR₆═CH—CH₂OH (phytol);
CH₂—CR₆H—CH₂—COOH (phytanic acid); or
CH₂—CR₆H—COOH (pristanic acid),
COOH (4,8,12-TMTD);
or which are a salt, an ester or an amide thereof, in particular chlorophyll.

4. A method according claim 1, characterised in that said PPAR/RXR heterodimer activator is phytanic acid, pristanic acid, TMTD (4,8,12-trimethyltridecanoic acid), a derivative of these acids or a combination thereof, the PPAR/RXR heterodimer activator being preferably phytanic acid and/or pristanic acid.

5. A method according to claim 1 characterised in that said product comprises phytol.

6. A method according to claim 1 characterised in that the livestock animals are slaughtered to produce said livestock product, in particular to produce skeletal meat, and the livestock animals are made to ingest said product for at least three days during the last week before the slaughtering.

7. A method according to claim 1 characterised in that the livestock animals are non-ruminant mammals or poultry, said product being given to the livestock animals so that a level of said PPAR/RXR heterodimer activator of at least 0.2%, preferably of at least 0.5% and more preferably of at least 1.0% of total FAME fatty acids (comprising a linear chain of at least 12 carbon atoms) is achieved in said livestock product, in particular in skeletal meat of the livestock animals, the non-ruminant mammals being preferably non-rodents.

8. A method according to claim 7, characterised in that said livestock animals are poultry and said livestock products eggs, said product being given to the livestock animals so that a level of said PPAR/RXR heterodimer activator of at least 1%, preferably of at least 3% and more preferably of at least 5% of total FAME fatty acid is achieved in egg yolk of said eggs.

9. A method according to claim 1 characterised in that the livestock animals are ruminants, said product being given to the livestock animals so that a level of said PPAR/RXR heterodimer activator of at least 0.7%, preferably of at least 0.9% and more preferably of at least 1.0% of total FAME fatty acid is achieved in skeletal meat of the livestock animals.

10. A method according to claim 1, characterised in that the livestock animals are ruminants, said product being given to the livestock animals so that a level of said PPAR/RXR heterodimer activator higher than 0.75%, preferably higher than 1.0% and more preferably higher than 1.5% of total FAME fatty acids is achieved in milk of the livestock animals.

11. A method according to claim 9 characterised in that said product comprises chlorophyll in a concentration of at least 0.25% by dry weight, preferably of at least 0.50% by dry weight and more preferably of at least 0.75% by dry weight.

12. A method according to claim 1, characterised in that the livestock animals are aquatic animals used to produce said livestock product in aquaculture, said product being given to the livestock animals so that a level of said PPAR/RXR heterodimer activator of at least 0.7%, preferably of at least 0.9% and more preferably of at least 1.0% of total long chain fatty acids is achieved in said livestock product.

13. A method according to claim 1, characterised in that said product is given at least once a day during said period of at least three days, said product being preferably given with the feed of the livestock animals.

14. A method according to claim 1, characterised in that said predetermined amount of said product contains at least 25×F meq., preferably at least 35×F meq., more preferably at least 50×F meq., and most preferably at least 65×F meq of said PPAR/RXR heterodimer activator and/or precursor thereof.

15. A method according to claim 1, characterised in that said predetermined amount of said product contains less than 175×F meq., and preferably less than 125×F meq of said PPAR/RXR heterodimer activator and/or precursor thereof.

16. A method according to claim 1, characterised in that for an initial trial period, during which the livestock animals are made to ingest said product, a parameter influenced by the ingestion of said product in at least a number of the individual livestock animals is determined and, after the initial trial period, the livestock animals are split up into at least two groups based on a difference in effect of said product on said parameter, the parameter which is determined being preferably the gain of weight and/or the feed intake of the individual livestock animals.

17. A method to supplement the human diet with a PPAR/RXR heterodimer activator, in which method livestock animals, used in agri- or aquaculture for producing a livestock product for human consumption, are made to ingest at least one product comprising said PPAR/RXR heterodimer activator and/or a precursor thereof which is metabolised by the livestock animals into said PPAR/RXR heterodimer activator, over such a period of time and in such an amount that the PPAR/RXR heterodimer activator is accumulated in the livestock animal so that an increased PPAR/RXR heterodimer activator level is achieved in the livestock product, characterised in that said PPAR/RXR heterodimer activator is phytanic acid, a metabolite of phytanic acid, a derivative of phytanic acid or of said metabolite, or a combination thereof and, in order to accumulate the PPAR/RXR heterodimer activator in the livestock animal, a predetermined amount of said product is given to the livestock animals over at least one period of at least three days, during which the livestock animals ingest a total amount of F kg feed dry weight, which predetermined amount of said product contains at least 5×F meq, preferably at least 10×F meq, and more preferably at least 15×F meq of said PPAR/RXR heterodimer activator and/or precursor thereof.

18. A method for improving the quality of carcass and meat of livestock animals, in particular of pigs, characterised in that the livestock animals are made to ingest at least one product which comprises a PPAR/RXR heterodimer activator selected from the group consisting of phytanic acid, metabolites of phytanic acid, and derivatives of phytanic acid and of said metabolite, and/or a precursor thereof which is metabolised by the livestock animals into said PPAR/RXR heterodimer activator, and, in order to achieve an improved skeletal meat quality, a predetermined amount of said product is given to the livestock animals over at least one period of at least three days, during which the livestock animals ingest a total amount of F kg feed dry weight, which predetermined amount of said product contains at least 5×F meq, preferably at least 10×F meq, and more preferably at least 15×F meq of said PPAR/RXR heterodimer activator and/or precursor thereof.

19. A method according to claim 18, characterised in that said product comprises phytol.

20. A livestock product for human consumption, in particular a livestock product for human consumption obtainable by a method according to claim 1, which live stock product comprises at least one PPAR/RXR heterodimer activator selected from the group consisting of phytanic acid, metabolites of phytanic acid and derivatives of phytanic acid and of said metabolites, characterised in that the livestock product comprising egg yolk of poultry eggs, which egg yolk has a level of said PPAR/RXR heterodimer activator of at least 1%, preferably of at least 3% and more preferably of at least 5% of total FAME fatty acids, the livestock product being in particular poultry eggs.

21. A livestock product according to claim 20, characterised in that the livestock product comprises at least 0.5 kg egg yolk.

22. A livestock product for human consumption, in particular a livestock product for human consumption obtainable by a method according to claim 1 which live stock product comprises at least one PPAR/RXR heterodimer activator selected from the group consisting of phytanic acid, metabolites of phytanic acid and derivatives of phytanic acid and of said metabolites, characterised in that the livestock product is skeletal meat of non-ruminant mammals or poultry having a fat content of maximum 70% on dry weight basis and a level of said PPAR/RXR heterodimer activator of at least 0.2%, preferably of at least 0.5% and more preferably of at least 1.0% of total FAME fatty acids.

23. A livestock product for human consumption, in particular a livestock product for human consumption obtainable by a method according to claim 1, which live stock product comprises at least one PPAR/RXR heterodimer activator selected from the group consisting of phytanic acid, metabolites of phytanic acid and derivatives of phytanic acid and of said metabolites, characterised in that the livestock product is skeletal meat of ruminants having a fat content of maximum 70% on dry weight basis and having a level of said PPAR/RXR heterodimer activator of at least 0.7%, preferably of at least 0.9% and more preferably of at least 1.0% of total FAME fatty acids.

24. A livestock product for human consumption, in particular a livestock product for human consumption obtainable by a method according to claim 1, which live stock product comprises at least one PPAR/RXR heterodimer activator selected from the group consisting of phytanic acid, metabolites of phytanic acid and derivatives of phytanic acid and of said metabolites, characterised in that the livestock product contains a mixture of milk of at least 8 ruminants having a level of said PPAR/RXR heterodimer activator higher than 0.75%, preferably higher than 1.0% and more preferably higher than 1.5% of total FAME fatty acids.

25. A livestock product for human consumption, in particular a livestock product for human consumption obtainable by a method according to claim 1, which live stock product comprises at least one PPAR/RXR heterodimer activator selected from the group consisting of phytanic acid, metabolites of phytanic acid and derivatives of phytanic acid and of said metabolites, characterised in that the livestock product are aquatic animals, in particular aquatic animals defined in the main group 4 “Fish and fish products” of the Europcode 2 version of Apr. 8, 1999, having a level of said PPAR/RXR heterodimer activator of at least 4.0%, preferably of at least 5.0%, more preferably of at least 6.0% and most preferably of at least 8.0% of total FAME fatty acids.

26. A feed for livestock animals for use in a method according to claim 1, characterised in that it comprises a PPAR/RXR heterodimer activator selected from the group consisting of phytanic acid, metabolites of phytanic acid, and derivatives of phytanic acid and of said metabolite, and/or a precursor thereof which is metabolised by the livestock animals into said PPAR/RXR heterodimer activator, the feed being composed to contain at least 5 meq/kg feed dry weight, preferably at least 10 meq/kg feed dry weight, and more preferably at least 15 meq/kg feed dry weight of said PPAR/RXR heterodimer activator and/or precursor thereof.

27. A feed according to claim 25, characterised in that it contains at least 5 meq/kg feed dry weight, preferably at least 10 meq/kg feed dry weight, and more preferably at least 15 meq/kg feed dry weight of phytol.
